(19) United States
(12) Patent Application Publication
(10) Pub. No.: US 2024/0285978 A1
(43) Pub. Date: Aug. 29, 2024

(54) MINIMALLY INVASIVE HISTOTRIPSY SYSTEMS AND METHODS

(71) Applicant: THE REGENTS OF THE UNIVERSITY OF MICHIGAN, Ann Arbor, MI (US)

(72) Inventors: Zhen XU, Ann Arbor, MI (US); Timothy Lewis HALL, Ann Arbor, MI (US); Greyson STOCKER, Ann Arbor, MI (US); Man ZHANG, Ann Arbor, MI (US)

(21) Appl. No.: 18/568,038

(22) PCT Filed: Apr. 7, 2022
(86) PCT No.: PCT/US2022/023784

§ 371 Date: Dec. 7, 2023

Related U.S. Application Data
(60) Provisional application No. 63/197,915, filed on Jun. 7, 2021.

(51) Int. Cl. A61N 7/00; A61B 8/00; A61B 8/12

(52) U.S. Cl. CPC A61N 7/00; A61B 8/12; A61B 8/4281; A61N 2007/0039; A61N 2007/0043

(57) ABSTRACT

Minimally invasive histotripsy systems and methods are provided. In some embodiments, a minimally invasive histotripsy device is inserted into a patient. The device can be inserted endoscopically through a natural orifice of the patient, or laparoscopically through an incision in the patient's skin. The minimally invasive histotripsy device can be advanced to the target tissue and acoustically coupled to the target tissue before applying histotripsy therapy to the target tissue. In some embodiments, the minimally invasive histotripsy device includes a transducer array with a focal length of approximately 10-40 mm, a diameter of less than 35 mm, and the ability to create a peak negative focal pressure of at least 20 MPa.

### PRIORITY CLAIM

This patent application claims priority to U.S. provisional patent application No. 63/197,915, titled “MINIMALLY INVASIVE HISTOTRIPSY SYSTEMS AND METHODS” and filed on Jun. 7, 2021, which is herein incorporated by reference in its entirety.

### STATEMENT AS TO FEDERALLY SPONSORED RESEARCH

This invention was made with government support under CA211217 awarded by the National Institutes of Health. The government has certain rights in the invention.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel histotripsy systems configured to produce acoustic cavitation, methods, devices and procedures for the minimally and non-invasive treatment of healthy, diseased and/or injured tissue. The histotripsy systems and methods described herein, also referred to Histotripsy, may include transducers, drive electronics, positioning robotics, imaging systems, and integrated treatment planning and control software to provide comprehensive treatment and therapy for soft tissues in a patient. More specifically, the present disclosure provides novel endoscopic histotripsy systems and associated endoscopic surgical procedures.

### BACKGROUND

Many medical conditions require invasive surgical interventions. Invasive procedures often involve incisions, trauma to muscles, nerves and tissues, bleeding, scarring, trauma to organs, pain, need for narcotics during and following procedures, hospital stays, and risks of infection. Non-invasive and minimally invasive procedures are often favored, if available, to avoid or reduce such issues. Unfortunately, non-invasive and minimally invasive procedures may lack the precision, efficacy or safety required for treatment of many types of diseases and conditions. Enhanced non-invasive and minimally invasive procedures are needed, preferably not requiring ionizing or thermal energy for therapeutic effect.

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, and high-intensity focused ultrasound (HIFU), Histotripsy relies on the mechanical action of cavitation for tissue destruction.

An important recent trend in medical interventions is a comprehensive drive towards less invasive yet effective procedures. Many disease states can now be addressed using minimally-invasive or non-invasive approaches, and many of these are performed under increasingly sophisticated imaging guidance. The progression from planar radiation therapy to stereotactic body radiation therapy (SBRT) is one such example, but radiation toxicity still limits treatment locations and volume. Thermal-based ablations are generally delivered percutaneously with imaging guidance, and include radiofrequency ablation, microwave ablation, and cryoablation. These technologies either heat or freeze targeted tissue which results in necrosis. All thermal modalities are impacted by the heat sink effect of blood flow, a critical reliance on physician expertise, tumor size, tumor location, and a lack of predictability of the ablation margins. High intensity focused ultrasound (HIFU) is a non-invasive ablation technique that uses externally applied ultrasound energy to cause thermal necrosis. HIFU has been used clinically to treat uterine fibroids, neurological diseases, and tumors in the prostate, breast, liver, and pancreas, but its clinical use is still infrequent due to anatomic challenges and long procedure times.

Histotripsy is a non-invasive focused ultrasound technology that uses ultrasound applied from outside the body and focused on a target tissue. The underlying mechanism of histotripsy is mechanical at the cellular level, which is entirely different from HIFU thermal therapy. The term histotripsy was coined at the University of Michigan in 2003. In Greek, “Histo” means “soft tissue,” and “tripsy” refers to breakdown. HIFU uses continuous or long exposure of ultrasound with intermediate applied pressure and high duty cycles (ultrasound ontime/total treatment time 10%) to heat target tissue. In contrast, histotripsy uses a low duty cycle (1%) to minimize heating, short ultrasound pulses (microseconds to milliseconds in length), and very high applied pressure to generate acoustic cavitation using endogenous gas in tissues. Acoustic cavitation is the initiation and dynamic changes of microbubbles activated by ultrasound. Histotripsy uses cavitation to mechanically break down and liquefy the target tissue into an acellular debris. Ultrasound imaging can be used to guide and monitor the histotripsy procedure in real time. In contrast to many existing minimally-invasive techniques, histotripsy can result in the non-invasive removal of tissue. When histotripsy is applied to a tissue-fluid interface (e.g., blood clots or cardiac tissue), tissue is eroded from the surface inwards, and eventually results in well-defined perforations. When targeting histotripsy to inside a bulk volume tissue (e.g., a tumor), histotripsy eventually liquifies the target tissue to an acellular homogenate, and the debris is absorbed over 1-3 months by the body, resulting in effective tissue removal.

The ability to effectively remove tissue allows histotripsy to be used in applications that are not possible with thermal techniques. The non-thermal nature also enables histotripsy to overcome many of the limitations associated with thermal devices (e.g., heat sink effect, lack of precise margins and predictability). Histotripsy has been investigated for many pre-clinical applications, including treatment for tumors in the liver, kidney, and prostate, neurological diseases, thrombosis, neonatal and fetal congenital heart disease, kidney stones, and biofilms. Phase I human trials have been undertaken for histotripsy treatment of benign prostatic hyperplasia and liver cancer, and early results suggest safety and feasibility in humans. This review provides a comprehensive overview of histotripsy, including the mechanism, bioeffects, parameters, instruments, preclinical and clinical studies, and advantages and limitations compared to related devices.

Minimally or non-invasive procedures, such as endoscopic procedures, are surgical techniques that use small incisions or natural orifices with less damage to the body than with traditional open surgery. It is associated with less pain, fewer complications and a shorter hospital stay. Minimally invasive surgical (MIS) techniques have been widely adopted over the last few decades. Among the reasons for this trend are a reduction in surgical complications, and reduced scarring. MIS techniques such as endoscopy, laparoscopy, and robotic surgery involve making small incisions or using natural orifices to insert surgical tools.

Histotripsy has been developed as a transcutaneous technique for treating cancer, neurological, and cardiovascular applications, including liver tumors, brain tumors, hemorrhagic stroke, and deep vein thrombosis. While a transcutaneous approach is promising for these applications, limited acoustic access (due to bony or gaseous structures) and deep overlying tissue can complicate transcutaneous histotripsy treatment of some organs, and sufficiently high pressure may not be achievable through treatment delivered from outside of the body. Examples of organs with limited acoustic access include the prostate (due to the pelvis and bowel), the heart and the pancreas (due to the ribcage and lungs).

Transcutaneous histotripsy transducers are large due to the need for a large amount of “active area” of piezoelectric material to generate the extremely high amplitude pressure pulses. For example, a transducer utilized for transcranial brain applications can have an aperture size of centimeters, while the transducer for liver treatment can have an aperture size of 14.7 centimeters.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1H illustrate an endoscopic ultrasound imaging and therapy system, including various embodiments of an ultrasound transducer array.

FIG. 2 is one embodiment of acoustically coupling a minimally invasive histotripsy device to a target tissue with a balloon catheter.

FIG. 3 is a method of treating tissue with a minimally invasive histotripsy device.

### SUMMARY OF THE DISCLOSURE

Histotripsy produces tissue fractionation through dense energetic bubble clouds generated by short, high-pressure, ultrasound pulses. When using pulses shorter than 2 cycles, the generation of these energetic bubble clouds only depends on where the peak negative pressure (P−) exceeds an intrinsic threshold for inducing cavitation in a medium (typically 26-30 MPa in soft tissue with high water content).

A method of performing a minimally invasive therapy on a target tissue, comprising the steps of inserting a minimally invasive histotripsy device into a patient, navigating the minimally invasive histotripsy device to the target tissue, identifying the target tissue with realtime imaging, acoustically coupling the minimally invasive histotripsy device to the target tissue, and applying histotripsy therapy to the target tissue with the minimally invasive histotripsy device to lyse at least a portion of the target tissue.

In some embodiments, the minimally invasive histotripsy device is inserted into the patient laparoscopically.

In other embodiments, the minimally invasive histotripsy device is inserted into the patient endoscopically.

In one example, the real-time imaging further comprises an ultrasound imaging transducer integrated into the minimally invasive histotripsy device.

In some embodiments, acoustically coupling further comprises applying an acoustic medium to the target tissue.

In another embodiment, acoustically coupling further comprises navigating a balloon catheter to the target tissue, filling the balloon catheter with an acoustic medium, and placing the balloon catheter in contact with the target tissue and the minimally invasive histotripsy device.

In one embodiment, applying histotripsy therapy further comprises generating a peak negative pressure of greater than 20 MPa in the target tissue.

In some embodiments, the target tissue comprises a prostate or a pancreas.

In some embodiments, the inserting step further comprises inserting the minimally invasive histotripsy device transrectally, laparoscopically, or via an open approach into the patient.

A minimally invasive histotripsy system is provided, comprising an elongate shaft configured to be inserted into a patient, a histotripsy therapy array disposed on a distal portion of the elongate shaft and being configured to apply histotripsy ultrasound pulses to a target tissue, and an ultrasound imaging transducer disposed on the distal portion of the device and being configured to image the target tissue in real time.

In some embodiments, the ultrasound imaging transducer is disposed centrally within the histotripsy therapy array.

In one embodiment, the histotripsy therapy array has an aperture size that is smaller than 4 cm.

In some implementations, the histotripsy therapy array is configured to produce a peak negative pressure of greater than 20 MPa.

In some examples, the histotripsy therapy array comprises four transducer elements. In one embodiment, the histotripsy therapy array comprises a flat array.

In some examples, the system further comprises a coupling device configured to acoustically couple the histotripsy therapy array to the target tissue.

In one embodiment, the coupling device comprises a balloon catheter configured to be inflated with an acoustic coupling medium.

### DETAILED DESCRIPTION

Provided herein are systems and methods that provide efficacious non-invasive and minimally invasive therapeutic, diagnostic and research procedures. In particular, provided herein are minimally invasive histotripsy systems and methods that provide targeted, efficacious histotripsy in a variety of different regions and under a variety of different conditions, particularly for target tissue regions that are difficult or impossible to treat with a transcutaneous histotripsy approach.

Systems and methods provided herein bypass the acoustic limitations for treatment of organs such as the prostate (due to the pelvis and bowel), the heart, and the pancreas, with a small, endoscopic histotripsy transducer that can be inserted into a small natural orifice (e.g., rectal insertion to access prostate, or esophageal insertion for cardiac ablation).

Balancing desired tissue destruction in target regions with the avoidance of damage to non-target regions presents a technical challenge. This is particularly the case where time efficient procedures are desired. Conditions that provide fast, efficacious tissue destruction tend to cause undue heating in non-target tissues. Under heating can be avoided by reducing energy or slower delivery of energy, both of which run contrary to the goals of providing a fast and efficacious destruction of target tissue. Provided herein are a number of technologies that individually and collectively allow for fast, efficacious target treatment without undesired damage to non-target regions.

The system, methods and devices of the disclosure may be used for the minimally or non-invasive acoustic cavitation and treatment of healthy, diseased and/or injured tissue, including in extracorporeal, percutaneous, endoscopic, laparoscopic, and/or as integrated into a robotically-enabled medical system and procedures. As will be described below, the histotripsy system may include various electrical, mechanical and software sub-systems, including a Cart, Therapy, Integrated Imaging, Robotics, Coupling and Software. The system also may comprise various Other Components, Ancillaries and Accessories, including but not limited to patient surfaces, tables or beds, computers, cables and connectors, networking devices, power supplies, displays, drawers/storage, doors, wheels, illumination and lighting and various simulation and training tools, etc. All systems, methods and means creating/controlling/delivering histotripsy are considered to be a part of this disclosure, including new related inventions disclosed herein.

In one embodiment, the histotripsy system is configured as a mobile therapy cart, which further includes a touchscreen display with an integrated control panel with a set of physical controls, a robotic arm, a minimally invasive histotripsy therapy device positioned or mounted on the distal end of the robot, and software to operate and control the system.

The mobile therapy cart architecture can comprise internal components, housed in a standard rack mount frame, including a histotripsy therapy generator, high voltage power supply, transformer, power distribution, robot controller, computer, router and modem, and an ultrasound imaging engine. The front system interface panel can comprise input/output locations for connectors, including those specifically for two ultrasound imaging probes (handheld and probe coaxially mounted in the therapy transducer), a histotripsy therapy transducer, AC power and circuit breaker switches, network connections and a foot pedal. The rear panel of the cart can comprise air inlet vents to direct airflow to air exhaust vents located in the side, top and bottom panels. The side panels of the cart include a holster and support mechanism for holding the handheld imaging probe. The base of the cart can be comprised of a cast base interfacing with the rack mounted electronics and providing an interface to the side panels and top cover. The base also includes four recessed casters with a single total locking mechanism. The top cover of the therapy cart can comprise the robot arm base and interface, and a circumferential handle that follows the contour of the cart body. The cart can have inner mounting features that allow technician access to cart components through access panels.

The touchscreen display and control panel may include user input features including physical controls in the form of six dials, a space mouse and touchpad, an indicator light bar, and an emergency stop, together configured to control imaging and therapy parameters, and the robot. The touchscreen support arm is configured to allow standing and seated positions, and adjustment of the touchscreen orientation and viewing angle. The support arm further can comprise a system level power button and USB and ethernet connectors.

The robotic arm can be mounted to the mobile therapy cart on arm base of sufficient height to allow reach and case of use positioning the arm in various drive modes into the patient/procedure work space from set up, through the procedure, and take down. The robotic arm can comprise six degrees of freedom with six rotating joints, a reach of 850 mm and a maximum payload of 5 kg. The arm may be controlled through the histotripsy system software as well as a 12 inch touchscreen polyscope with a graphical user interface. The robot can comprise force sensing and a tool flange, with force (x, y, z) with a range of 50 N, precision of 3.5 N and accuracy of 4.0 N, and torque (x, y, z) with a range of 10.0 Nm, precision of 0.2 Nm and accuracy of 0.3 Nm. The robot has a pose repeatability of +/−0.03 mm and a typical TCP speed of 1 m/s (39.4 in/s). In one embodiment, the robot control box has multiple I/O ports, including 16 digital in, 16 digital out, 2 analog in, 2 analog out and 4 quadrature digital inputs, and an I/O power supply of 24V/2A. The control box communication comprises 500 Hz control frequency, Modbus TCP, PROFINET, ethernet/IP and USB 2.0 and 3.0.

In some embodiments, the robotic arm can hold a minimally invasive histotripsy device, such as a laparoscopic histotripsy device or an endoscopic histotripsy device. In some embodiments, movement of the minimally invasive histotripsy device can be controlled with the robotic arm. In other embodiments, the arm serves only has a support or holder for the minimally invasive histotripsy device, and the minimally invasive histotripsy procedure can be instead performed manually by a physician or surgeon.

The minimally invasive histotripsy device can include an ultrasound transducer array signed and configured for minimally invasive insertion into a patient. For example, the transducer array can be configured to be inserted into an endoscope or cannula to access a patient. In some embodiments, the transducer array is disposed on a distal end of a shaft, which can then be inserted into the endoscope or the cannula. In other embodiments, the transducer array can be disposed on a distal end of a flexible catheter. In some embodiments, the transducer array can have a cross section small enough to fit within an endoscope, laparoscope, or other surgical cannula or minimally invasive access device. For example, the minimally invasive device can have a cross-sectional length smaller than 4 cm. In some embodiments, the transducer array can have a cross sectional length smaller than 50 mm, smaller than 40 mm, smaller than 30 mm, or smaller than 20 mm.

Endoscopic histotripsy has broad potential for clinical applications, using upper gastrointestinal (GI), transvaginal, transrectal approaches or bronchoscopic approaches. For example, transesophageal treatment of pancreatic cancer and peri-pancreatic walled-off necrosis; transvaginal/transrectal treatment of prostatic hyperplasia (BPH), prostate cancer, and common symptomatic female pelvic conditions, such as uterine leiomyomas and deep infiltrating endometriosis (DIE). Additional abdominopelvic applications include endometrial or cervical lesion resection, uterine tissue sampling, chronic abscess or retroperitoneal fibrosis breakdown, and lymph node dissection. In the airways and lungs, histotripsy may be used for treatment of lung tumors or pulmonary lymph nodes, and/or for locally removing unwanted tissue that may be involved with, or adjacent to, critical structures (e.g., pulmonary artery).

In some embodiments, ultrasound imaging transducers can be included on the minimally invasive histotripsy device. For example, in one embodiment, a transducer array can include both therapy transducers and ultrasound imaging transducers. The therapy transduces can be configured to provide histotripsy therapy to tissue, and the ultrasound imaging transducers can be configured to provide ultrasound images of the target tissue and/or the histotripsy therapy in real-time.

The system software and work-flow can be configured to allow users to control the system through touchscreen display and the physical controls, including but not limited to, ultrasound imaging parameters and therapy parameters. The graphical user interface of the system comprises a work-flow based flow, with the general procedure steps of 1) registering/selecting a patient, 2) inserting the minimally invasive histotripsy probe into the patient through a natural orifice or an small insertion, 3) planning, comprising imaging the patient (and target location/anatomy) with the freehand imaging probe, and robot assisted imaging with the transducer head for final gross and fine targeting, including contouring the target with a target and margin contour, of which are typically spherical and ellipsoidal in nature, and running a test protocol (e.g., test pulses) including a bubble cloud calibration step, and a series of predetermined locations in the volume to assess cavitation initiation threshold and other patient/target specific parameters (e.g., treatment depth), that together inform a treatment plan accounting for said target's location and acoustic pathway, and any related blockage (e.g., tissue interfaces, bone, etc.) that may require varied levels of drive amplitude to initiate and maintain histotripsy. Said parameters, as measured as a part of the test protocol, comprising calibration and multi-location test pulses, are configured in the system to provide input/feedback for updating bubble cloud location in space as needed/desired (e.g., appropriately calibrated to target cross-hairs), as well as determining/interpolating required amplitudes across all bubble cloud treatment locations in the treatment volume to ensure threshold is achieved throughout the volume. Further, said parameters, including but not limited to depth and drive voltage, may be also used as part of an embedded treatability matrix or look up table to determine if additional cooling is required (e.g., off-time in addition to time allocated to robot motions between treatment pattern movements) to ensure robust cavitation and intervening/collateral thermal effects are managed (e.g., staying below t43 curve for any known or calculated combination of sequence, pattern and pathway, and target depth/blockage). The work-flow and procedure steps associated with these facets of planning, as implemented in the system software may be automated, wherein the robot and controls system are configured to run through the test protocol and locations autonomously, or semi-autonomously. Following planning, the next phase of the procedure work-flow, 3) the treatment phase, is initiated following the user accepting the treatment plan and initiating the system for treatment. Following this command, the system is configured to deliver treatment autonomously, running the treatment protocol, until the prescribed volumetric treatment is complete. The status of the treatment (and location of the bubble cloud) is displayed in real-time, adjacent to various treatment parameters, including, but not limited to, of which may include total treatment time and remaining treatment time, drive voltage, treatment contours (target/margin) and bubble cloud/point locations, current location in treatment pattern (e.g., slice and column), imaging parameters, and other additional contextual data (e.g., optional DICOM data, force torque data from robot, etc.). Following treatment, the user may use the minimally invasive histotripsy device, and subsequently, the freehand ultrasound probe to review and verify treatment, as controlled/viewed through the system user interface. If additional target locations are desired, the user may plan/treat additional targets, or dock the robot to a home position on the cart if no further treatments are planned.

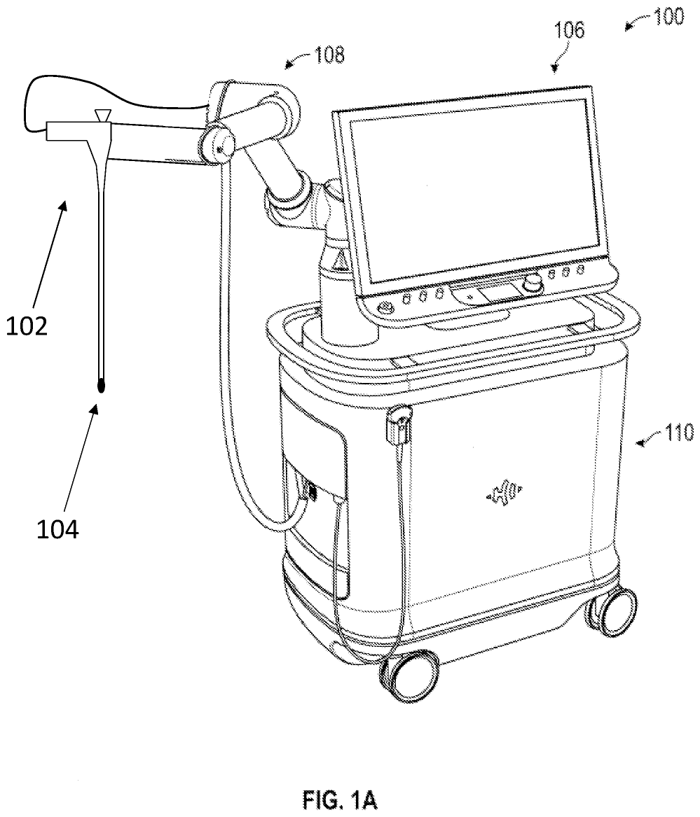

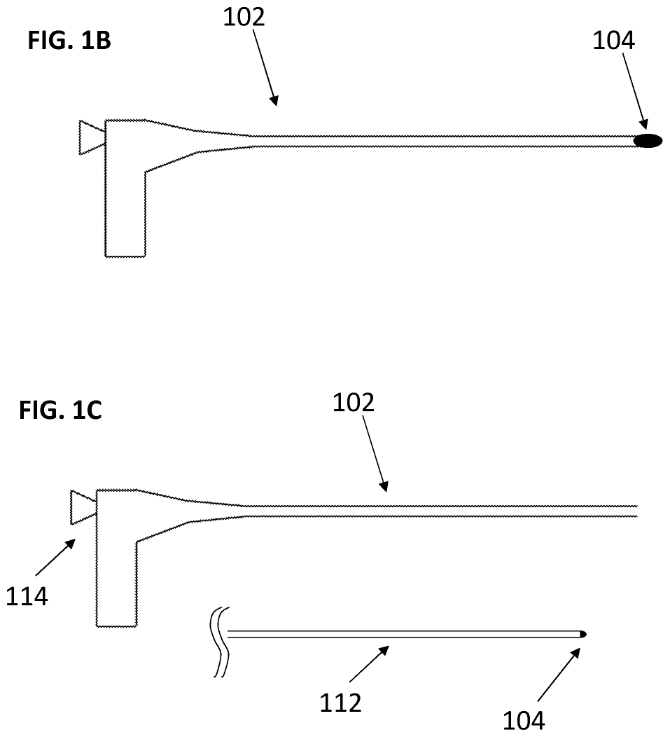

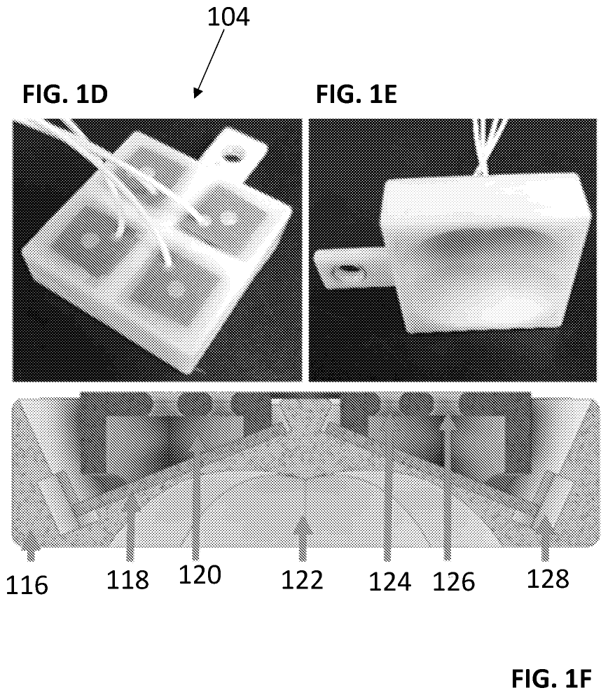

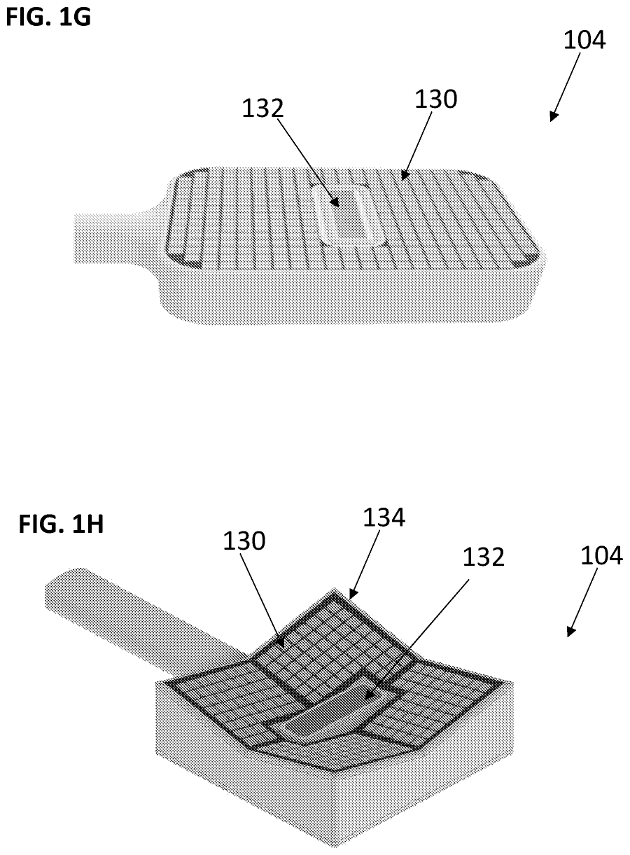

FIG. 1A generally illustrates histotripsy system 100 according to the present disclosure, comprising minimally invasive histotripsy device 102, an ultrasound transducer array 104 (which optionally includes an imaging system such as ultrasound imaging transducers), a display and control panel 106, a robotic positioning arm 108, and a cart 110. The system can further include a standalone imaging system, such as ultrasound, CT, MRI, etc., not shown.

FIG. 1B is one embodiment of a minimally invasive histotripsy device 102 which can include an ultrasound transducer array 104. In the embodiment of FIG. 1B, the ultrasound transducer array 104 is disposed on a distal end of the minimally invasive device. Referring to FIG. 1C, however, the transducer array 104 can be disposed on a distal end of a shaft or catheter 112, which can then be configured to be inserted into the minimally invasive device 102 via access port 114. In this embodiment, the target tissue can be approached with the minimally invasive device, and then the transducer array can be safely navigate through a lumen of the minimally invasive device to the target tissue without damaging the transducer array.

As described above, the ultrasound transducer array can include one or more therapy transducers and/or one or more ultrasound imaging transducers. In some embodiments, the transducer array comprises only therapy transducers, and imaging is performed separately (e.g., with a separate ultrasound imaging device). In some embodiments, imaging system can be positioned in the center of the therapy transducers. However, other embodiments can include the imaging system positioned in other locations within the therapy transducer, or even directly integrated into the therapy transducer. In some embodiments, the imaging system is configured to produce real-time imaging at a focal point of the therapy transducer.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with cross-sectional imaging (e.g., CT, cone beam CT and/or MRI scanning). In other embodiments, it may be arranged for use in an operating room and a sterile environment, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., chest, abdomen, pelvis, head and neck, and extremities, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g. compatible within cone beam CT work-space for collecting imaging data pre, peri and/or post histotripsy).

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with at least a single negative/tensile phase sufficient to cause a cluster of bubble nuclei intrinsic to the medium to undergo inertial cavitation, 2) Shock-Scattering Histotripsy: Delivers typically pulses 3-20 cycles in duration. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

This disclosure describes minimally invasive histotripsy system and associated endoscopic/laparoscopic/minimally invasive surgical procedures. In contrast to high frequency ((≥5 MHZ) histotripsy devices with very small focal zone and precise focal treatment, the main features of the minimally invasive histotripsy system described in this disclosure are a lower frequency and the capability for volume ablation at a reasonable speed. Histotripsy requires extremely high focal pressure (peak negative pressure p->20 MPa). A minimally invasive endoscopic ultrasound transducer requires a small transducer aperture size. The pressure output decreases with the decreasing size of the transducer aperture and decreasing frequency. A minimally invasive endoscopic histotripsy system with a sufficient small aperture (<4 cm) and low frequency (≤2 MHZ) has not been considered or realized prior to this disclosure.

Higher frequency histotripsy (≥5 MHZ) results in a higher focal gain, enabling a high focal pressure with a small aperture transducer. However, the volume of the transducer focal zone (focal volume) decreases with increasing frequency. For example, at 1 MHZ, the focal volume is typically an elliptical volume of ˜1×1×3 mm, while at 5 MHz, the focal volume is typically ˜0.2×0.2×0.6 mm. To treat a target volume with a high frequency, the transducer focus must be moved so that multiple focal volumes are stacked together to the treat desired shape and volume. It takes many more overlapping focal zones and thus much longer time to treat the same target volume using a higher frequency transducer such as 5 MHz compared to a lower frequency transducer such as 1 MHz. Therefore, a lower frequency histotripsy pulse sequence (≤2 MHZ) is provided in this disclosure and enables treatment of a target volume within a reasonable time.

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Transducer Array and Fabrication

A key component of histotripsy therapy is a high-power focused ultrasound transducer array configured to deliver sufficiently high ultrasound pressure and power to generate cavitation in the target tissue. Traditional transducer fabrication techniques include heating a large piece of piezoelectric (PZT) or piezoceramic composite (PCC) material and shaping the material to the appropriate curved shape with high mechanical precision. Next, the shaped PZT or PCC material can be cut into individual transducer elements. Electrode connections are then soldered to the individual transducer elements. In some implementations, a thin curved matching layer can then be bonded to the curved PZT or PCC transducer elements.

The transducer arrays provided in this disclosure are much smaller than those used for traditional transcutaneous histotripsy. For example, the aperture size of minimally invasive histotripsy transducer arrays as provided herein are smaller than 4 cm to allow insertion into a natural orifice, a minimally invasive access device, or a small incision in the patient's skin. As histotripsy requires very high pressure, the minimally invasive histotripsy transducer array with a small aperture can still produce a peak negative pressure >20 MPa.

The central frequency of the minimally invasive histotripsy transducer arrays described herein can have a lower central frequency (≤2 MHZ) in contrast to the higher frequency transducers typically used for high intensity focused ultrasound (HIFU) endoscopic transducers. A lower frequency is purposely selected to achieve a sufficiently fast ablation speed, as mentioned above.

To achieve a high focal pressure >20 MPa using a small aperture transducer with a lower frequency (≤2 MHZ), a combination of piezoelectric material, matching layer, and electric driver needs to be carefully selected to maximize the pressure output.

For example, in one specific embodiment, transducer modules can be constructed that include a single flat, 1 cm piezoelectric in a waterproof housing. The transducer itself can operate at a central frequency of (≤2 MHZ), for example, 1 MHZ. Two piezoelectric materials were chosen for testing: a high strength porous PZT (PZ36, Meggitt-Ferroperm, Kvistgaard, Denmark) and a standard hard PZT ceramic (SM111, Steiner & Martins Inc, Miami, Florida). Both of these materials have been successfully used in the past for fabrication of histotripsy systems. Matching layers can include Somos PerFORM (DSM Functional Materials, Elgin, Illinois) which is a nanoparticle filled 3D printed plastic with a relatively high acoustic impedance and sound speed (5.04 MRayl, 3150 m/s), a two-layer strategy incorporating aluminum and FR-4 fiberglass, and a concave acoustic lens, printed out of PerFORM with a focal length selected based on the target tissue (e.g., 25 mm). All matching layers (other than the lens) can be designed to be ¼ wavelength in thickness. The ¼ wavelength thickness is desirable to maximize ultrasound transmission efficacy through a matching layer of the probe to the skin.

In another embodiment, referring to FIGS. 1D-1F, a four element transducer array 104 is provided. FIG. 1D is a back view of the transducer array and housing, FIG. 1E is a front view of the housing without any transducer elements, and FIG. 1F is a cross-sectional view showing all the transducer array components, including a housing 116, transducer elements 118, a clamping piece 120, a compound lens 122, a cable routing hole 124, an epoxy backfilling port 126, and an accommodation 128 for a front solder connection. In one example, the transducer array can have a working distance greater than 15 mm and an aperture size less than 35 mm. One specific embodiment includes four flat 1.5 MHz transducer elements with individually focused elements and 3D printed acoustic lenses. To keep the aperture size below 35 mm, each element can be a 13 mm square in size.

In another embodiment, referring to FIG. 1G, the transducer array 104 can comprise a flat array transducer with a plurality of small flat element modules 130. A flat array as shown can be configured to maximize the electric focal steering range while simplifying acoustic coupling with a target tissue. A flat array can allow coupling to a relatively flat tissue surfaces within the body with only an acoustic coupling medium (e.g., an acoustic gel), thereby not requiring a balloon catheter for coupling as described above. Furthermore, flat arrays have a high electric focal steering range, although a large number of elements is required to achieve electric focusing. The flat module design can greatly simply and streamline transducer array fabrication. Referring still to the transducer array of FIG. 1G, the array can further include an integrated imaging transducer or transducer array 132. As shown, this imaging transducer is disposed in a center of the array 104, but in other embodiments the imaging transducer can be located in different positions on or within the array.

In another embodiment, shown in FIG. 1H, transducer array 104 can be constructed featuring multiple planar modules 134 and an imaging transducer or array 132. Each module can be constructed featuring a plurality of elements 130, arranged in a 2D fashion, featuring a common electrical connection on one side of the elements. The transducer itself can operate at any of the parameters described herein. In one embodiment, the transducer can have a central frequency of (≤2 MHZ), for example, 1 MHz. Electronic focal steering can also be used to focus the acoustic fields of the elements, and to manipulate the location of the focus. This design simplifies fabrication, and also increases steerability over spherically focused arrays.

It is understood that improvements on piezoelectric material, matching layer material, and electronics are advancing constantly. Thus, the selection of the combination described above is based on the available resources at a given time, but the objective remains the same, and similar testing and selection process will be used.

Minimally invasive histotripsy transducer arrays as described herein typically have a focal distance 2-6 cm to allow enough working distance (from the front surface of the transducer) <5 cm that can reach relative shallow targets for minimally invasive surgical use. The geometric design of the endoscopic transducer is based on the desired surgical application. The size of the transducer is determined to fit in the natural orifice or the minimally invasive access device. The focal distance is determined to cover the depth range of the target tissue from the surface of the natural orifice or access device.

The minimally invasive histotripsy transducer array can be a single focused ultrasound transducer. However, a phased or linear focused array transducer with electric focal steering capability can significantly increase the treatment speed. As such, the histotripsy focal zone can be moved via electronic focal steering to cover the target tissue volume. The mechanical and electronic focal steering can also be combined together to increase the size of the target volume that can be treated.

### Acoustic Coupling

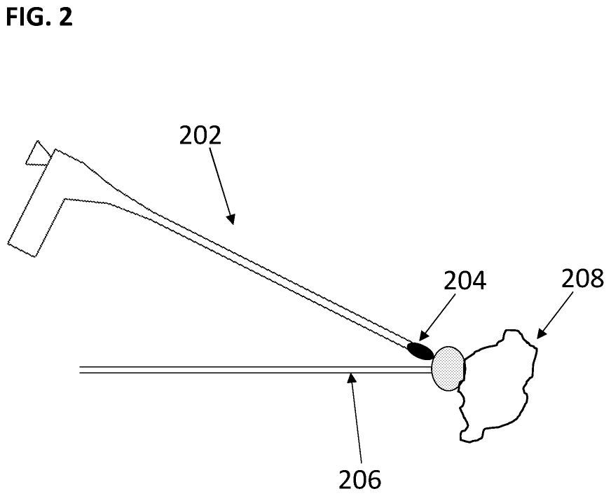

Acoustic coupling is necessary to acoustically couple the histotripsy therapy transducer(s) to the target tissue. For transcutaneous applications, an acoustic coupling gel or medium can be applied directly to the patient's skin, and the therapy transducer can be applied to the acoustic coupling medium. For minimally invasive procedures, however, applying an acoustic coupling medium or gel directly to the area of treatment is not always a viable option. In one embodiment, referring to FIG. 2, a balloon catheter 206 can be advanced to a target tissue 208, and the balloon catheter can be filled with an acoustic coupling medium such as saline, water, or acoustic coupling gel. The balloon catheter can then be placed into contact with both the target tissue and a transducer array 204 of a minimally invasive histotripsy device 202 to provide acoustic coupling. Minimally invasive histotripsy can be performed on the target tissue, before withdrawing both the minimally invasive histotripsy device and the balloon catheter. In some embodiments, the balloon of the balloon catheter is inflated or filled to a size that enables proper spacing between the transducer array 204 and the target tissue 208. For example, if the transducer array has a focal distance of 6 cm, then the balloon catheter can be expanded to a 6 cm diameter to treat a surface lesion or surface target tissue site within the patient's body. Similarly, if the target volume is, for example, at a depth of 2 cm from a surface of an organ such as a liver, and the focal distance of the transducer array is 6 cm, then the balloon can be inflated or filled to a 4 cm diameter to place the focus of the transducer array on that target tissue of the liver when the balloon contacts the surface of the liver and the transducer array contacts the other side of the balloon.

### Surgical Methods

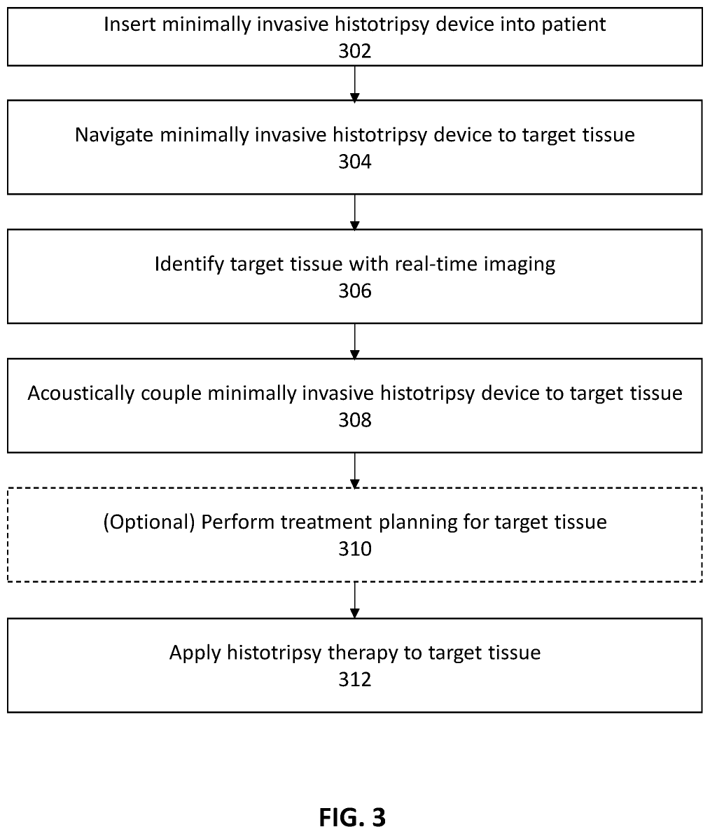

Surgical methods of using minimally invasive histotripsy devices, as discussed above, are provided herein. Referring to the flowchart of FIG. 3, at step 302, a minimally invasive histotripsy device can be inserted into the body of a patient. As described above, the minimally invasive histotripsy device can include or be integrated into an endoscope, a laparoscope, or any other minimally invasive access device. In the example of an endoscope, the endoscope can be inserted into a natural orifice of the patient (e.g., a mouth). In the example of a laparoscope, a small incision can be made in the patient's skin and the laparoscope can be inserted into the patient through the incision. In some embodiments, the minimally invasive histotripsy device is integrated into the endoscope/laparoscope, and in other embodiments, the minimally invasive histotripsy device including the transducer array can be inserted into a lumen of the access device.

At step 304 of FIG. 3, the minimally invasive histotripsy device can be navigated to the target tissue. In some embodiments, the access device, such as the endoscope or laparoscope, includes navigation or steerable functionality that allows the access device to gain access to the target tissue. In other embodiments, the minimally invasive histotripsy device itself has navigation/steerable functionality and can be further used to navigate within the patient's body to access the target tissue.

Next, at step 306 of FIG. 3, the target tissue can be identified with real-time imaging. In some embodiments, the real-time imaging is integrated within the minimally invasive histotripsy device, as described above. In some embodiments, the real-time imaging can comprise 2D or even 3D ultrasound imaging. For example, a series of 2D B-mode ultrasound images of the target volume can be obtained with the real-time imaging, by either rotating the imaging transducer(s) or linearly moving the imaging transducer(s) with a robotic arm of the system. Multiple scans can be obtained to re-construct a 3D ultrasound image of the target tissue. In some embodiments, a planned outline of the target tissue volume can be drawn on each of the 2D ultrasound image frames. Alternatively, the planned outline of the target tissue volume can be drawn on the reconstructed 3D ultrasound image.

Next, at step 308 of FIG. 3, the minimally invasive histotripsy device can be acoustically coupled to the target tissue. In some embodiments, the transducer array can be directly coupled to the target tissue with an acoustic medium, such as an acoustic gel. However, this type of coupling is not feasible for all target tissue types. In other embodiments, acoustic coupling can be achieved by navigating a balloon catheter between the minimally invasive histotripsy device and the target tissue, and inflating the balloon catheter with an acoustic medium such as saline, water, or an acoustic gel.

Optionally, at step 310, additional treatment planning can be performed for the target tissue. For example, in one embodiment, the histotripsy focal zone can be marked on the ultrasound image(s). A grid of treatment points separated by preset spacings between focal zones can then be created to cover the outlined planned target volume with multiple overlapping focal zones. Once the treatment grid is generated, short bursts of testing histotripsy pulses can be applied to points at the boundary of the planned target volume to generate transient cavitation that can be viewed under real-time imaging. If there is a difference between the planned and actual cavitation locations, the treatment grid can be recalibrated accordingly. The histotripsy testing locations and the cavitation generated as a result can also be used to adjust the driving parameters of the transducer array to ensure that the desired focal pressures are achieved during therapy.

At step 312, histotripsy therapy can be applied to the targeted with the minimally invasive histotripsy device. In some embodiments, the therapy can be delivered under real-time imaging, as described above. As mentioned above, the minimally invasive histotripsy device can include a transducer array with a size less than 4 cm, a working distance or focal length larger than 10 mm, and must be configured to generate a peak negative pressure p->20 MPa to be able to produce sufficient cavitation in the target tissue to ablate or lyse the target tissue. The histotripsy focus can be scanned over the target tissue volume by mechanically moving the focus, or electric focal steering, or a combination of mechanical and electric focal steering. After treatment, the real-time imaging can be used to evaluate the ablation volume.

### Transrectal Prostate Treatment

In one specific embodiment, the minimally invasive histotripsy device can be inserted into a rectum of the patient. The histotripsy transducer array can then be navigated to the prostate of the patient. To be able to access and treat the prostate transrectally, the minimally invasive histotripsy device can have a working distance or focal length of 10-40 mm and be able to produce a peak negative focal pressure of at least 30 MPa, with a total aperture size of less than 35 mm.

### Pancreas Treatment

In another embodiment, the minimally invasive histotripsy device can be used to treat a pancreas of a patient through a laparoscopic or open approach. The histotripsy transducer array can then be navigated to the pancreas of the patient. To be able to access and treat the pancreas with an open or laparoscopic approach, the minimally invasive histotripsy device can have a working distance or focal length of 10-40 mm and be able to produce a peak negative focal pressure of at least 30 MPa, with a total aperture size of less than 35 mm.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of performing a minimally invasive therapy on a target tissue, comprising the steps of:
inserting a minimally invasive histotripsy device into a patient;
navigating the minimally invasive histotripsy device to the target tissue;
identifying the target tissue with real-time imaging;
acoustically coupling the minimally invasive histotripsy device to the target tissue; and
applying histotripsy therapy to the target tissue with the minimally invasive histotripsy device to lyse at least a portion of the target tissue.

2. The method of claim 1, wherein the minimally invasive histotripsy device is inserted into the patient laparoscopically.

3. The method of claim 1, wherein the minimally invasive histotripsy device is inserted into the patient endoscopically.

4. The method of claim 1, wherein the real-time imaging further comprises an ultrasound imaging transducer integrated into the minimally invasive histotripsy device.

5. The method of claim 1, wherein acoustically coupling further comprises applying an acoustic medium to the target tissue.

6. The method of claim 1, wherein acoustically coupling further comprises:
navigating a balloon catheter to the target tissue;
filling the balloon catheter with an acoustic medium; and
placing the balloon catheter in contact with the target tissue and the minimally invasive histotripsy device.

7. The method of claim 1, wherein applying histotripsy therapy further comprises generating a peak negative pressure of greater than 20 MPa in the target tissue.

8. The method of claim 1, wherein the target tissue comprises a prostate.

9. The method of claim 8, wherein the inserting step further comprises inserting the minimally invasive histotripsy device transrectally into the patient.

10. The method of claim 1, wherein the target tissue comprises a pancreas.

11. The method of claim 10, wherein the inserting step further comprises inserting the minimally invasive histotripsy device laparoscopically into the patient.

12. A minimally invasive histotripsy system, comprising:
an elongate shaft configured to be inserted into a patient;
a histotripsy therapy array disposed on a distal portion of the elongate shaft and being configured to apply histotripsy ultrasound pulses to a target tissue; and
an ultrasound imaging transducer disposed on the distal portion of the device and being configured to image the target tissue in real time.

13. The minimally invasive histotripsy system of claim 12, wherein the ultrasound imaging transducer is disposed centrally within the histotripsy therapy array.

14. The minimally invasive histotripsy system of claim 12, wherein the histotripsy therapy array has an aperture size that is smaller than 4 cm.

15. The minimally invasive histotripsy system of claim 12, wherein the histotripsy therapy array is configured to produce a peak negative pressure of greater than 20 MPa.

16. The minimally invasive histotripsy system of claim 12, wherein the histotripsy therapy array comprises four transducer elements.

17. The minimally invasive histotripsy system of claim 12, wherein the histotripsy therapy array comprises a flat array.

18. The minimally invasive histotripsy system of claim 12, further comprising a coupling device configured to acoustically couple the histotripsy therapy array to the target tissue.

19. The minimally invasive histotripsy system of claim 12, wherein the coupling device comprises a balloon catheter configured to be inflated with an acoustic coupling medium.
